(19) United States
(12) Patent Application Publication
(10) Pub. No.: US 2021/0265029 A1
(43) Pub. Date: Aug. 26, 2021

(54) GENERATING MICROBIOME FINGERPRINTS, DIETARY FINGERPRINTS, AND MICROBIOME ANCESTRY

(71) Applicant: Zoe Global Limited, London (GB)

(72) Inventors: Nicola Segata, Trento (IT); Richard James Davies, London (GB); Jonathan Thomas Wolf, London (GB)

(73) Assignee: Zoe Global Limited

(21) Appl. No.: 17/179,179

(22) Filed: Feb. 18, 2021

Related U.S. Application Data
(60) Provisional application No. 62/979,932, filed on Feb. 21, 2020.

(51) Int. Cl. G16H 10/60; G16H 20/60; G16H 10/40

(52) U.S. Cl. CPC G16H 10/60; G16H 10/40; G16H 20/60

(57) ABSTRACT

Techniques are disclosed herein for generating microbiome fingerprints, dietary fingerprints, and microbiome ancestry. Microbiome data associated with an individual is analyzed to generate a microbiome fingerprint, and a dietary fingerprint for a user. A “microbiome fingerprint” uniquely identifies the microbiome of a user at a particular point in time and is based on a combination of different profiles generated from the microbiome data. The dietary fingerprint identifies how the microbiome of a user is associated with one or more different indexes, such as a dietary index and/or a particular characteristic (e.g., a Mediterranean diet index, a vegetarian diet index, a fast food index, an internal fat index, a fat-digesting index, a carbohydrate-digesting index, a health index, a fasting index, a ketogenic index, . . . ). The microbiome data may also be utilized to determine microbiome ancestry for the user that indicates other users to which the user is related to.

### CROSS-REFERENCE TO RELATED APPLICATIONS

This application claims priority to U.S. Provisional Patent Application No. 62,979,932 entitled “Generating Microbiome Fingerprints, Dietary Fingerprints, and Microbiome Ancestry,” filed Feb. 21, 2020, which is incorporated herein by reference in its entirety.

### BACKGROUND

Today, individuals can measure a large number of health characteristics without having to go to a lab or clinic. For example, individuals may obtain an analysis of their microbiome by mailing a sample, collected at home, to a company for analysis. Generally, a microbiome analysis includes determining the composition and function of a community of microbes in a particular location, such as within the gut of an individual. A microbiome of the gut is made up of trillions of microorganisms, such as bacteria, and their genetic material that live in the intestinal tract, including bacteria, archaea or archeobacteria, viruses, and microeukaryotes.

These microorganisms appear to be an important part of digesting food, assisting with absorbing and synthesizing nutrients, regulating metabolism, body weight, and immune regulation, as well as contributing to regulating brain functions and mood. Microbiomes of different individuals, however, vary greatly. For instance, it is estimated that only ten to thirty percent of the bacterial species in a microbiome is common across different individuals. Much of this diversity of microbiomes remains unexplained, yet diet, environment, and host genetics appear to play a part. Determining how to utilize the results of the microbiome analysis, however, can be challenging.

### BRIEF DESCRIPTION OF THE DRAWINGS

FIG. 1 is a block diagram depicting an illustrative operating environment in which microbiome data is analyzed to generate microbiome fingerprints, dietary fingerprints, and microbiome ancestry for users.

FIG. 2 is a block diagram depicting an illustrative operating environment in which a data ingestion service receives, and processes test data associated with at home tests and sample collections.

FIG. 3 is a flow diagram showing a process illustrating aspects of a mechanism disclosed herein for obtaining and utilizing microbiome data for a user to generate microbiome fingerprints, dietary fingerprints, and microbiome ancestry for users.

FIG. 4 is a flow diagram showing a process illustrating aspects of a mechanism disclosed herein for generating a microbiome fingerprint for a user.

FIG. 5 is a flow diagram showing a process illustrating aspects of a mechanism disclosed herein for generating a dietary fingerprint for a user.

FIG. 6 is a flow diagram showing a process illustrating aspects of a mechanism disclosed herein for generating a microbiome ancestry for a user.

FIG. 7 is a flow diagram showing a process illustrating aspects of a mechanism disclosed herein for obtaining test data, including microbiome data, that may be utilized for generating microbiome fingerprints, dietary fingerprints, and microbiome ancestry for users.

FIG. 8 is a computer architecture diagram showing one illustrative computer hardware architecture for implementing a computing device that might be utilized to implement aspects of the various examples presented herein.

### DETAILED DESCRIPTION

The following detailed description is directed to technologies for generating microbiome fingerprints, dietary fingerprints, and microbiome ancestry. Using the technologies described herein, microbiome data associated with an individual and other data are analyzed to generate a microbiome fingerprint, a dietary fingerprint, and microbiome ancestry data for a user. As used herein, a “microbiome fingerprint” is data that uniquely identifies the microbiome of a user at a particular point in time, and a “dietary fingerprint” is data that identifies how the microbiome of a user at a particular point in time is associated with one or more different indexes associated with a diet and/or health characteristics. The indexes may include, but are not limited to a Mediterranean diet index, a vegetarian diet index, a fast food index, an internal fat index, a fat-digesting index, a carbohydrate-digesting index, a health index, a fasting index, a gut transit time index, a ketogenic index, and the like. According to some configurations, one or more computers of a microbiome service generate a score, such as from 0-100, (or some other indicator) that indicates how closely the microbiome of the user is associated with a particular index.

As an example, the Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low post-prandial triacylglycerol (TAG) rises. The carbohydrate-digesting index score indicates how closely the microbiome of the user resembles someone with low post-prandial glucose rises. The health index score indicates how closely the microbiome of the user resembles someone that is healthy. The fasting index score indicates how closely the microbiome of the user resembles someone that fasts regularly. The gut transit time index indicates gut function including variance of the microbiome and appears to be predictive of postprandial lipid and glucose responses and visceral fat in users that are healthy. The ketogenic index score indicates how closely the microbiome of the user resembles someone who is ketogenic.

The microbiome service may utilize the microbiome data generated from a microbiome sample and/or other data to generate a microbiome fingerprint, dietary fingerprint, and/or microbiome ancestry data for a user. For example, the microbiome service may perform an analysis of the microbiome data associated with a microbiome sample to identify the microbial composition (e.g., the species, genes, taxa, and the like). In some examples, some/all of the analysis of the microbiome service may be performed by a service provider that is external from the microbiome service. The microbiome service may obtain this portion of the microbiome data from the external service provider(s). The microbiome service may also generate reconstructed microbial genomes, determine a diversity of the microbiome, identify functions of the microbiome, identify a uniqueness of the microbiome, identify interesting species, and the like. According to some configurations, the microbiome data may include gut transit time. For example, the gut transit time may be the time between ingestion of a food including food coloring paste, such as royal blue (or some other color), and the first time the user sees the appearance of the color of the food coloring in their stool.

In some examples, the microbiome data of the user is utilized with other data that is gathered about the user, as well as other users. For instance, users may provide responses to questionnaires, data about food that is eaten, sleep habits, and the like. Among other uses, this other data in addition to the microbiome data may be utilized to assist in determining a “microbiome ancestry” of a user. A “microbiome ancestry” for a user indicates that the user has relationships with other users and/or locations based on a similarity of the microbiome data (e.g., the microbiome fingerprint) for a particular user with other users.

In some examples, the microbiome service generates a microbiome ancestry by analyzing the microbiome data of the user and determining how closely the microbiome of the user is related to other users, and/or locations. For instance, the microbiome service may determine a number of other users to which the microbiome of the user is most closely related to. In some configurations, the microbiome service compares the microbiome data, such as the microbiome fingerprint, of the user to microbiome data, such as the microbiome fingerprints, of other users to determine whether the user is related to any of the other users.

As briefly discussed, the microbiome service may also identify one or more locations to which the microbiome of the user is associated with. For example, the microbiome service may identify the countries the microbiome of the user is associated with (e.g. 75% North America, 25% Mexico). This identification may be based on microbiome data of users at different locations and/or different populations (e.g., English, American, French, Mexican, Italian, . . . ). For instance, the microbiome service may determine that the microbiome fingerprint of the user is more similar to a microbiome of a user in France even though the user is from England.

According to some configurations, a user may “opt-in” to allow use of the microbiome data and/or other data associated with a user. In some examples, the user “opts-in” to participate in a social network and/or some other communication mechanism to discuss issues related to the microbiome data such as a microbiome ancestry (e.g., compare diets and background with other users). The microbiome service may also compare the microbiome of the user with other family members, and/or other users when the users have “opted-in” to allow this. For instance, the microbiome service may identify how many strains they share (with respect to sharing with unrelated persons) and overall how similar they are compared to the average.

In some examples, the microbiome service may provide a user interface (UI), such as a Graphical User Interface (GUI) for a user to view and interact with microbiome data and/or other data associated with the microbiome fingerprints, dietary fingerprints, and microbiome ancestry. For instance, the GUI may display microbiome fingerprint data that shows various characteristics of the microbiome fingerprint, dietary fingerprint data that shows various characteristics of the dietary fingerprint, microbiome ancestry data that shows various characteristics of the microbiome ancestry, recommendation data that identifies one or more recommendations relating to changing the microbiome of the user, and the like. In some examples, the UI/GUI may display information relating to determining a time a food was consumed to the time the user first saw the appearance of the color (e.g., blue) within a stool. In some examples, the date and time (hours, minutes) of the blue appearance is automatically recorded within a mobile application after a user selected a UI element (e.g., clicked on the associated UI button).

As an example, the microbiome service may provide recommendations to increase the diversity of foods eaten as there is no one good food for a healthy microbiome. The recommendations may include to eat different gut-healthy foods, eat fermented foods, minimize highly processed foods (things like emulsifiers and artificial sweeteners may affect the microbiome). The microbiome service may base the recommendations on data obtained from the user, and other users.

The microbiome service may also track the state of the microbiome of the user over time. For example, the microbiome service may provide data related to different microbiome analysis. In this way, the user may see how changes made by the user (e.g., eating different foods, changing exercise patterns, . . . ) have affected the microbiome.

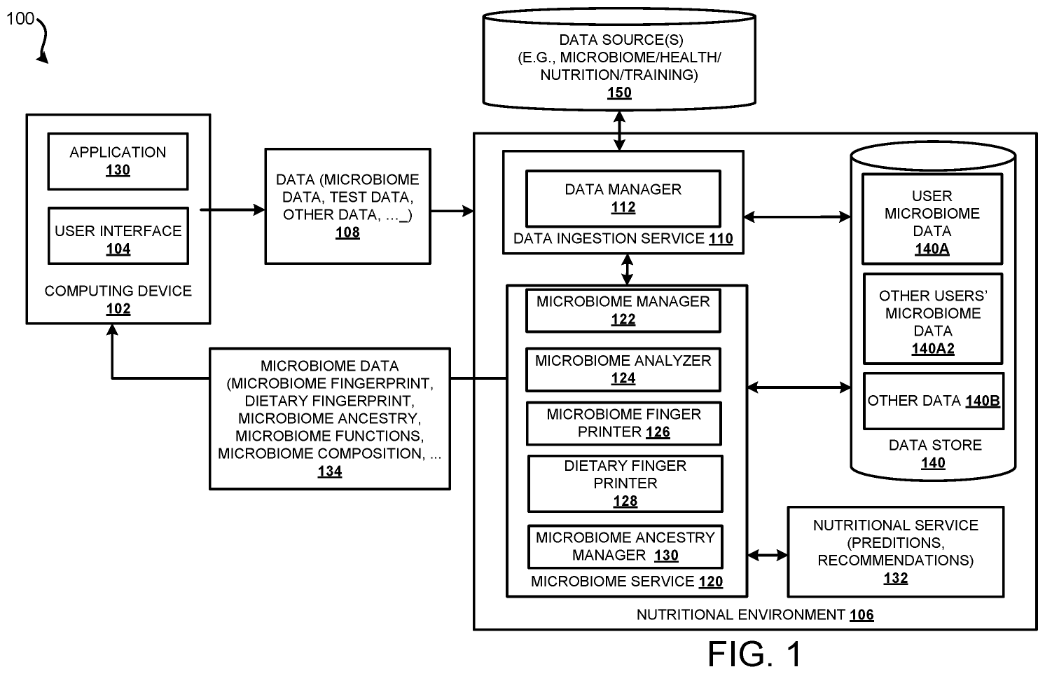

Additional details regarding the various components and processes described above relating to generating microbiome fingerprints, dietary fingerprints, and microbiome ancestry will be presented below with regard to FIGS. 1-8.

It should be appreciated that the subject matter presented herein may be implemented as a computer process, a computer-controlled apparatus, a computing system, or an article of manufacture, such as a computer-readable storage medium. While the subject matter described herein is presented in the general context of program modules that execute on one or more computing devices, those skilled in the art will recognize that other implementations may be performed in combination with other types of program modules. Generally, program modules include routines, programs, components, data structures and other types of structures that perform particular tasks or implement particular abstract data types.

Those skilled in the art will also appreciate that aspects of the subject matter described herein may be practiced on or in conjunction with other computer system configurations beyond those described herein, including multiprocessor systems, microprocessor-based or programmable consumer electronics, minicomputers, mainframe computers, handheld computers, personal digital assistants, e-readers, mobile telephone devices, tablet computing devices, special-purposed hardware devices, network appliances and the like.

In the following detailed description, references are made to the accompanying drawings that form a part hereof, and that show, by way of illustration, specific examples or examples. The drawings herein are not drawn to scale. Like numerals represent like elements throughout the several figures (which may be referred to herein as a “FIG.” or “FIGS.”).

FIG. 1 is a block diagram depicting an illustrative operating environment 100 in which microbiome data is analyzed to generate microbiome fingerprints, dietary fingerprints, and microbiome ancestry for users. An individual, such as an individual interested in obtaining microbiome fingerprints, dietary fingerprints, and microbiome ancestry information, may communicate with the nutritional environment 106 using a computing device 102 and possibly other computing devices, such as mobile electronic devices.

In some configurations, an individual may generate and provide data 108, such as microbiome data, test data, and/or other data. According to some examples, the user may utilize a variety of at home biological collection devices, which collect a biological sample. These devices may include but are not limited to “At Home Blood Tests” which use blood extraction devices such as finger pricks which in some examples are used with dried blood spot cards, button operated blood collection devices using small needles and vacuum to collect liquid capillary blood and the like. In some examples there may be home biological collection devices such as a stool test which is then assayed to produce biomarker test data such as gut microbiome data.

A computing device, such as a mobile phone or a tablet computing device can also be used to improve the accuracy of the measurements. For instance, instead of relying on an individual to accurately record the time a test was taken, or a sample was obtained, the computing device 102 can record information that is associated with the event. The computing device 102 may also be utilized to capture the timing data associated with the test (e.g., the time the test was performed, . . . ), or the sample was collected, and provide that data to a data ingestion service 110. As an example, a clock (or some other timing device) of the computing device 102 may be used to record the time the measurement(s) were collected and/or samples were obtained.

As illustrated in FIG. 1, the operating environment 100 includes one or more computing devices 102, in communication with a nutritional environment 106. In some examples, the nutritional environment 106 may be associated with and/or implemented by resources provided by a service provider network such as provided by a cloud computing company. The nutritional environment 106 includes a data ingestion service 110, a microbiome service 120, a nutritional service 132, and a data store 140. The nutritional service 132 can be utilized to generate personalized nutritional recommendations. For example, the personalized nutritional recommendations can be generated using techniques described in U.S. patent application Ser. No. 15/894,798, filed on Feb. 12, 2018, which is incorporated by reference herein in its entirety. According to some examples, the nutritional service 132 may provide recommendations based on the microbiome fingerprint, dietary fingerprint, microbiome ancestry data and/or other data.

The nutritional environment 106 may include a collection of computing resources (e.g., computing devices such as servers). The computing resources may include a number of computing, networking and storage devices in communication with one another. In some examples, the computing resources may correspond to physical computing devices and/or virtual computing devices implemented by one or more physical computing devices.

It should be appreciated that the nutritional environment 106 may be implemented using fewer or more components than are illustrated in FIG. 1. For example, all or a portion of the components illustrated in the nutritional environment 106 may be provided by a service provider network (not shown). In addition, the nutritional environment 106 could include various Web services and/or peer-to-peer network configurations. Thus, the depiction of the nutritional environment 106 in FIG. 1 should be taken as illustrative and not limiting to the present disclosure.

The data ingestion service 110 facilitates submission of data utilized by the microbiome service 120 and, in some configurations, the nutritional service 132. Accordingly, utilizing a computing device 102, an electronic collection device, an at home biological collection device or via in clinic biological collection, an individual may submit data 108 to the nutritional environment 106 via the data ingestion service 110. Some of the data 108 may be sample data, biomarker test data, and some of the data 108 may be non-biomarker test data such as photos, barcode scans, timing data, and the like.

A “biomarker” or biological marker generally refers to one or more measurable indicators (that may be combined using various techniques) of some biological state or condition associated with an individual. Stated another way, a biomarker may be anything that can be used as an indicator of particular disease, state or some other physiological state of an organism. A biomarker can typically be measured accurately (either objectively and/or subjectively) and the measurement is reproducible (e.g., blood glucose, triglycerides, insulin, c-peptides, ketone body ratios, IL-6 inflammation markers, hunger, fullness, and the like).

The measured biomarkers can include many different types of health data such as microbiome data which may be referred to herein as “microbiome data”, blood data, glucose data, lipid data, nutrition data, wearable data, genetic data, biometric data, questionnaire data, psychological data (e.g., hunger, sleep quality, mood, . . . ), objective health data (e.g., age, sex, height, weight, medical history, . . . ), as well as other types of data. Generally, “health data” can refer to any psychological, subjective and/or objective data that relates to and is associated with one or more individuals. The health data might be obtained through testing, self-reporting, and the like. Some biomarkers change in response to eating food, such as blood glucose, insulin, c-peptides and triglycerides and their lipoprotein components.

To understand the differences in nutritional responses for different users, dynamic changes in biomarkers caused by eating food such as a standardized meal (“post-prandial responses”) may be measured. By understanding an individual's nutritional responses, in terms of blood biomarkers such as glucose, insulin and triglycerides levels, or non-blood biomarkers such as the microbiome, a nutritional service may be able to choose the food that is more suited for that particular person.

Data may also be obtained by the data ingestion service 110 from other data sources, such as data source(s) 150. For example, the data source(s) 150 can include, but are not limited to microbiome data associated with one or more users, nutritional data (e.g., nutrition of particular foods, nutrition associated with the individual, and the like), health data records associated with the individual and/or other individuals, and the like.

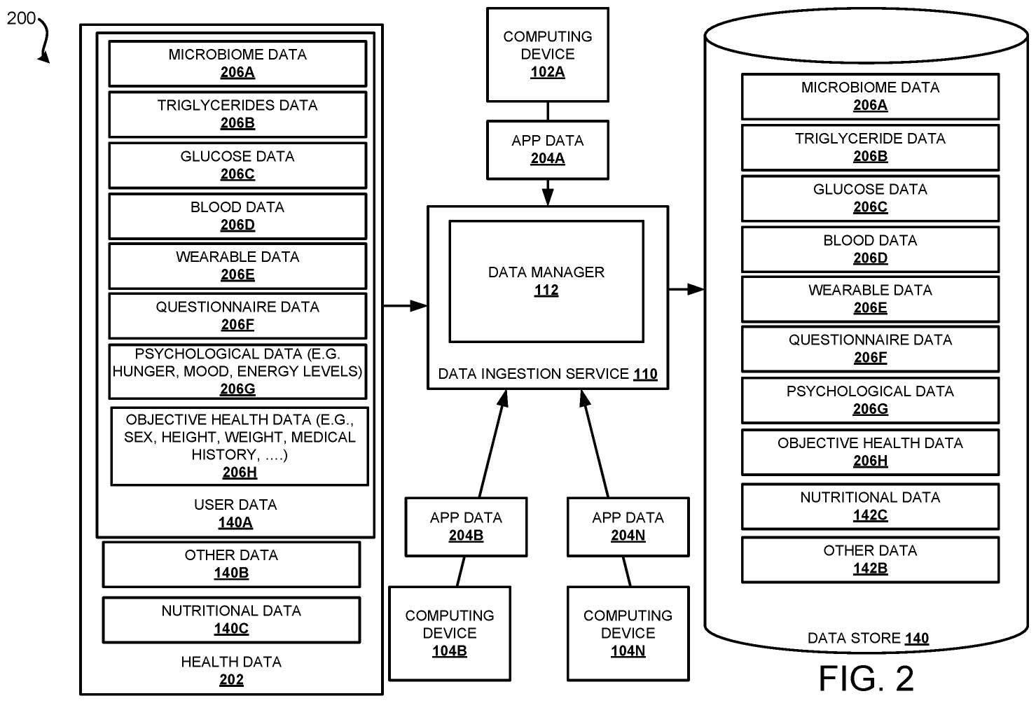

The data, such as data 108, or data obtained from one or more data sources 150, may then be processed by the data manager 112 and/or the microbiome manager 122 and included in a memory, such as the data store 140. As illustrated, the data store 140 can be configured to store user microbiome data 140A, other users' microbiome data 140A2, and other data 140B (See FIG. 2 for more details on the data ingestion service 110). In some examples, the user microbiome data 140A and other users' microbiome data 140A2 includes microbiome data.

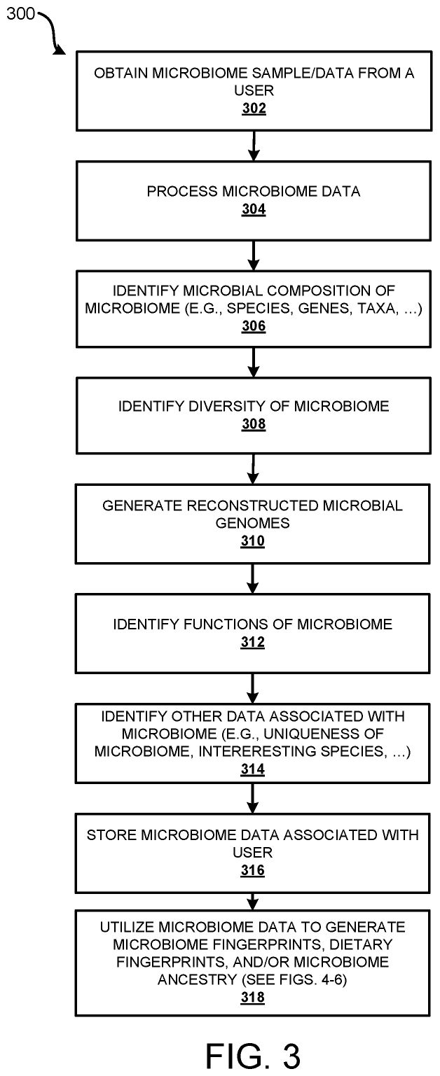

As discussed in more detail below (See FIGS. 3-7 for more details), the microbiome service 120 utilizing the microbiome manager 122, the microbiome analyzer 124, the microbiome finger printer 126, the microbiome dietary finger printer 128, and the microbiome ancestry manager 130, analyzes the data 108 associated with a user and generate a microbiome fingerprint, a dietary fingerprint, and microbiome ancestry data for the user. According to some configurations, the microbiome service 120 utilizes both data 108 associated with the user and data from other users.

In some examples, the microbiome manager 122 may utilize one or more machine learning mechanisms. For example, the microbiome manager 122 can use a classifier to classify the microbiome within a classification category (e.g., associate with a particular dietary index, a geographic location, . . . ). In other examples, the microbiome manager 122 may use a scorer to generate scores that may provide an indication of the dietary index associated with a user, how closely related the user is to other users based on the microbiome data, and the like.

The data ingestion service 110 and/or the microbiome service 120 can generate one or more user interfaces, such as a user interface 104 and/or user interface 104B, through which an individual, utilizing the computing device 102, or some other computing device, may provide/receive data from the nutritional environment 106. For example, the data ingestion service 110 may provide a user interface 104 that allows an individual of the computing device 102A to submit data 108 to the nutritional environment 106.

In some cases, the individual can also provide biological samples to a lab for testing, using a biological collection device. According to some configurations, this will include At Home Blood Tests. According to some configurations, individuals can provide a sample for microbiome analysis. As an example, metagenomic testing can be performed using the sample to allow the DNA of the microbes in the microbiome of an individual to be digitalized. Generally, a microbiome analysis includes determining the composition and functional potential (here called just “function”) of a community of microbes in a particular location, such as within the gut of an individual. An individual's microbiome appears to have a strong relationship to metabolism, weight and health, yet only ten to thirty percent of the bacterial species in a microbiome is estimated to be common across different individuals. Techniques described herein combine different techniques to assist in improving the accuracy of the data captured outside of a clinical setting, such as calculating accurate glucose responses to individual meals, which can then be linked to measures like the microbiome.

According to some configurations, individuals can provide a sample or samples of their stool for microbiome analysis as part of the at home biological collection. In some cases, this sample may be collected without using a chemical buffer. The sample can then be used to culture live microbes, or for chemical analysis such as for metabolites or for genetic related analysis such as metagenomic or metatranscriptomic sequencing. In such cases it may suffer from changes in microbial composition due to causes including microbial blooming from oxygen in the period between being collected and when it is received in the lab, where it will be immediately assayed or frozen. In some cases, to avoid this change in bacterial composition after collection, the sample may be frozen at low temperatures very rapidly after collection. The sample can then be used to culture live bacteria, or for chemical analysis or for metagenomic sequencing. This collection can be done as part of an in clinic biological collection or at home where the collection kit is configured to deliver such low temperatures and maintain them until a courier has taken the sample to a lab.

A stool sample may be combined with a chemical preservation buffer such as ethanol as part of the at home collection process to stop further microbial activity, which allows a sample to be kept at room temperature before being received at the lab where the assay is done. In some examples, the buffer may be a proprietary chemical product sold and validated by other companies for the task of freezing the microbial activity while still allowing the sample to be processed for metagenomics sequencing. A buffer allows for such a sample to be posted in the mail without issues of microbial blooming or other continuing changes in microbial composition. The buffer may however prevent some biochemical analyses from being done, and because preservation buffers are likely to kill a large fraction of the microbial population it is unlikely that samples conserved in preservation buffers can be used for cultivation assays. In some cases, a user may do multiple stool tests over time, so that one can measure changes in the microbiome over time, or measure changes in the microbiome in response to meals, or changes in the microbiome in response to other clinical or lifestyle variations.

In some examples, the stool sample may be collected using a scoop or swab from a stool that is collected by the user using a stool collection kit that prevents the stool from falling into a toilet. Because there is a very high microbial load in the gut microbiome compared, for example, to the skin microbiome it is also possible that in some cases the stool sample is taken from paper that is used to clean the user's behind after they have passed a stool. This is only possible if the quantity of stool is large enough that the microbes from the stool greatly exceed the microbes that will be picked up from the user's skin or environmental contaminants. In any of these cases the scoop, swab or tissue may be placed inside a vial that contains a buffer solution. If the user then ensures the stool comes into contact with the buffer for example by shaking then this stops further microbial activity and allows the solution to be kept at room temperature without a significant change in microbial composition. In some cases, a sterile synthetic tissue can be used that does not have biological origins such as paper, so that when the DNA of the sample is extracted there is no contamination from the tissue. According to some examples, the tissue can be impregnated with a liquid to help capture more stool from the user's skin, where the liquid does not interfere with the results of the stool test and is not potentially dangerous for the human body.

In some cases, the timing and quality of the stool sample can be recorded using the computing device 102, for example using a camera. Where there are multiple stool tests the computing device 102 can use a barcode (or some other identifier) to confirm the timing and identity of that particular sample. Other data can also be collected. For example, data about how the sample was stored, how long the sample was stored before being supplied to the lab for analysis, and the like.

While the data ingestion service 110, the microbiome service 120, the nutritional service 132 are illustrated separately, all or a portion of these services may be located in other locations or together with other components. For example, the data ingestion service 110 may be located within the microbiome service 120. Similarly, the microbiome manager 122 may be part of a different service, and the like.

According to some examples, some individuals may be asked to visit a clinic to combine at home data with data collected at a clinic. The purpose of the clinic visit is to allow much higher accuracy of measurement for a subset of the individual's data, which can then be combined with the lower-quality at home data. This may be used by the microbiome service 120 to improve the quality of the at home data.

According to some examples, the day before the visit to the clinic, the individuals are asked to avoid taking part in any strenuous exercise and to limit the intake of alcohol. In some configurations, the microbiome service 120 can analyze the data 108, such as data obtained from an activity tracker, to determine whether the individual followed the instructions of avoiding strenuous exercise. Similarly, the nutritional service 132, or some other device or component, may analyze the foods eaten by the individual by analyzing food data that indicates the foods eaten by the user. Individuals may be provided with instructions for the tests (e.g., avoid eating high fat or high fiber meals that may interfere with test results, fasting, drinking water, . . . ).

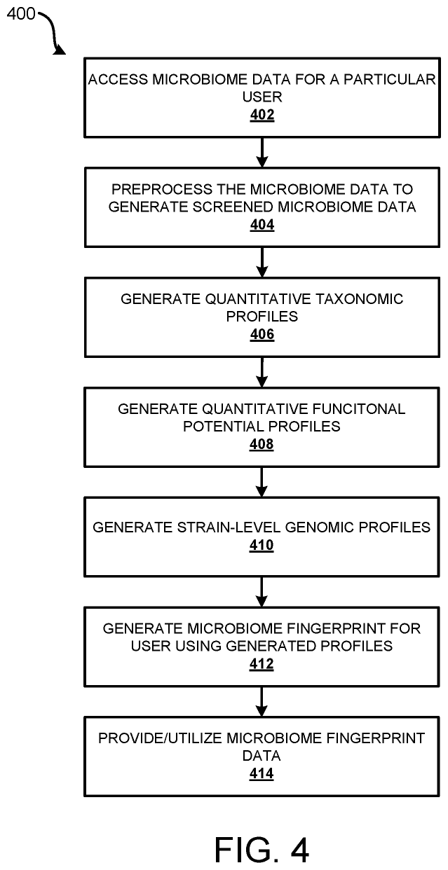

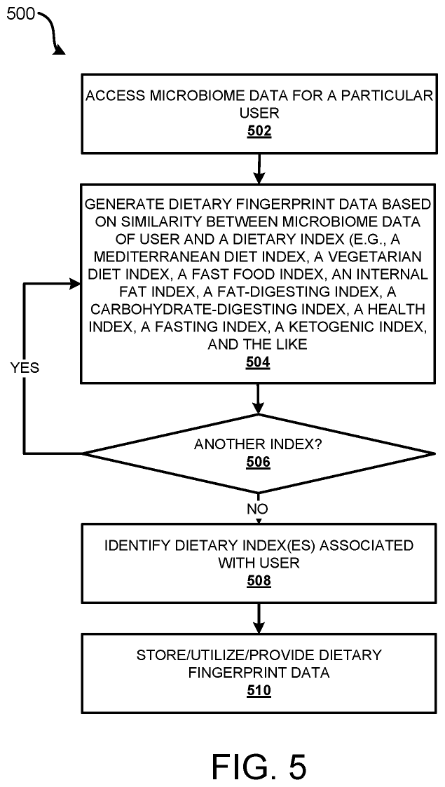

As described in more detail below with regard to FIGS. 4 and 5, the microbiome service 120 may use the microbiome manager 122 to generate a microbiome fingerprint, and a dietary fingerprint for a user. As discussed above, a “microbiome fingerprint” is data that uniquely identifies the microbiome of a user at a particular point in time. According to some configurations, the microbiome finger printer 126 generates a microbiome fingerprint from a user based on different profiles generated from the microbiome data, such as but not limited to quantitative taxonomic profiles, quantitative functional potential profiles, and strain-level genomic profiles. In some examples, the profiles are generated by the microbiome finger printer 126 and/or the microbiome analyzer 124.

According to some configurations, the microbiome fingerprint is a combination of descriptors, including, but not limited to (1) the quantitative (i.e. relative abundance) taxonomic profiles (i.e., the names or generally identifiers (IDs) in case of unknown entities of microbial species or other taxonomic units), (2) the quantitative (i.e. relative abundance) functional potential profiles, (i.e., the names or generally identifiers (IDs) in case of unknown entities of microbial gene families, microbial pathways, and microbial functional modules), and (3) the strain-level genomic profiles (i.e., the reconstruction of the genomes or part of the genomes of as many microbes present in the microbiome as possible).

The microbiome fingerprint may be generated by the microbiome finger printer 126 using various techniques and methods. In some configurations, generation of the microbiome fingerprint includes obtaining the microbiome sample, generating DNA from the sample, preprocessing the raw sequencing data to the generate quality-screened sequencing data, and transforming the sequencing data is transformed into the numerical and genomics sets for the descriptors utilized to generate the microbiome fingerprint (e.g., quantitative taxonomic profiles, quantitative functional potential profiles, and strain-level genomic profiles).

The microbiome analyzer 124 may also be configured to perform processing associated with the microbiome data. For example, the microbiome analyzer 124 may be configured to generate and/or process sequencing data associated with the microbiome of the user. See FIG. 4 for more details on generating the profiles. After generating the profiles, the microbiome finger printer 126 may generate the microbiome fingerprint for the user. In some examples, the dietary finger printer 128 combines the data associated with the different profiles generated.

In some configurations, the microbiome service 120 may be used to process gut transit time data associated with different users. As discussed above, the gut transit time indicates gut function for a user including variance of the microbiome of the user. The gut transit time may also be used by the microbiome service 120 to predict postprandial lipid and glucose responses and visceral fat in users that are healthy.

According to some examples, the UI 104 may display information to a user relating to determining a time a food was consumed that included food coloring, (or some other marker) to the time the user first saw the appearance of the color (e.g., blue) within an excreted stool. For example, the food may be injected with a blue dye (or possibly another color), which may be referred to herein as the “blue dye method” before being consumed by a user. Using the blue dye method provides benefits over other transit time measures (e.g., scintigraphy, wireless motility capsule, radiopaque markers, and breath testing) since it is inexpensive and scalable. For example, specialized equipment and staff are not required for the blue dye method. Further, gut transit time, measured via the blue dye method, is a more informative marker of gut microbiome function than traditional measures of stool consistency and frequency.

In some examples, the date and time (hours, minutes) of the appearance of the coloring (or other marker) is automatically recorded by the microbiome service 120, or some other device or component, after a user selected a UI element (e.g., clicked on the associated UI button) or some other triggering event (e.g., recognition within image data of the color of the dye within the stool). In some configurations, the user or some other device/component (e.g., image processing) may also identify/record stool frequency and/or consistency. For instance, the user, or some device/component, may record how often they have a bowel movement and record the consistency (e.g., using the Bristol Stool Form (BSF) scale). Generally, a lower BSF scale score corresponds to longer gut transit time.

The microbiome service 120 may use the gut transit time to generate a classification for the diversity of the microbiome for the user. According to some examples, the microbiome service 120 may select from different classifications (e.g., two, three, four, . . . ) based on the gut transit time for the user. For instance, the microbiome service 120 may select from four different classifications (e.g., a fastest classification of less than 14 hours, a fast normal classification between 14 hours and 38 hours, a slower normal classification between 38 hours and 58 hours, and a slow classification of greater than 58 hours) depending on the gut transit time for the user. More or less classifications may be used. Gut microbiome composition predicts gut transit time classes and longer gut transit time are linked with Akkermansia muciniphila, Bacteroides, and Alistipes spp relative abundances

The gut transit time may provide a good indication of variation in the gut microbiome, in terms of both relative abundance and alpha diversity. Alpha diversity reflects how diverse a single microbiome sample is, using a measure of richness (i.e., number of species detected) and the Shannon index, which accounts for both evenness and abundance. As such, the classification for the user based on a gut transit time may be used as an informative marker of gut function and may be used to predict postprandial lipid and glucose responses and visceral fat.

The dietary finger printer 128 is configured to generate a dietary fingerprint for the user. As discussed above, the “dietary fingerprint” of a user indicates how the microbiome of a user is associated with one or more different indexes that may be associated with a particular diet and/or a health characteristic. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like.

According to some configurations, the dietary finger printer 128 generates a score for each of the different indexes, such as from 0-100, (or some other indicator) to indicate how closely the microbiome of the user is associated with a particular index. For example, the dietary finger printer 128 may generate a score for each of the indexes based on how closely the microbiome of the user resembles a typical microbiome of someone that is known to follow a specific diet. For example, a score of 100 may indicate that the diet is strongly correlated to a particular diet, a score of 0 would indicate no correlation, and a score between 0 and 100 would indicate a different correlation. According to some configurations, the dietary finger printer 128 generates a Mediterranean diet index score, a vegetarian diet index score, a fast food index score, an internal fat index score, a fat-digesting index score, a carbohydrate-digesting index score, a health index score, and the like.

The Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low post-prandial triacylglycerol (TAG) rises. The carbohydrate-digesting index score indicates how closely the microbiome of the user resembles someone with low post-prandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic.

In other configurations, the dietary finger printer 128, or some other service or component may utilize different mechanisms to determine whether the microbiome of the user resembles a particular diet and/or group. For instance, the dietary finger printer 128 may utilize a machine learning mechanism to classify the microbiome of the user within a classification and/or generate a score, or some other indicator that indicates how closely the microbiome data of the user matches the microbiome data of a representative user associated with the particular index.

The microbiome ancestry manager 130 is configured to generate microbiome ancestry data for a user. A “microbiome ancestry” refers to microbiome data that indicates that the user has relationships with other users and/or locations. In some examples, the microbiome service analyzes the microbiome data of the user and determines how closely the microbiome of the user is related to other users, and/or locations. For instance, the microbiome service may determine a number of other users to which the microbiome of the user is most closely related to. In some configurations, the microbiome ancestry manager 130 compares the microbiome data of the user to microbiome data of other users to identify a relationship. Similar to generating the scores for the different indexes performed by the dietary finger printer 128, the microbiome ancestry manager 130 may generate a score for each comparison between the user and the other users. The scores that indicate a close relationship (e.g., above a specified value) with the user may be identified as related.

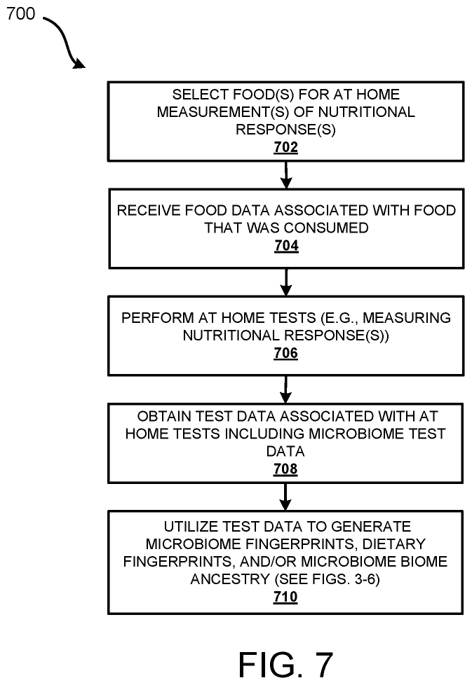

As briefly discussed, the microbiome service may also identify one or more locations to which the microbiome of the user is associated with. For example, the microbiome service may identify the countries the microbiome of the user is associated with (e.g. 75% North America, 25% Mexico). This identification may be based on microbiome data of users at different locations and/or different populations (e.g., English, American, French, Mexican, Italian, . . . ). See FIG. 7 for additional details for generating the microbiome ancestry data.

The microbiome analyzer 124, or some other device or component, may analyze the microbiome data of a user before/after generating the microbiome fingerprint, dietary fingerprint, and/or microbiome ancestry for a user. For example, the microbiome analyzer 124 may perform an analysis of the microbiome data to identify the microbial composition of the microbiome (e.g., the species, genes, taxa, and the like). The microbiome service may also generate reconstructed microbial genomes, determine a diversity of the microbiome, identify functions of the microbiome, identify a uniqueness of the microbiome, identify interesting species, and the like.

In some examples, the microbiome data of the user is compared (e.g., by the microbiome service 120) with other data that is gathered about the user, as well as other users. For instance, users may provide responses to questionnaires, data about food that is eaten, sleep habits, and the like. Among other uses, this data may be utilized to determine a “microbiome ancestry” of a user.

In some examples, the microbiome service may provide a user interface (UI), such as a Graphical User Interface (GUI) 104 for a user to view and interact with data associated with the microbiome fingerprints, dietary fingerprints, and microbiome ancestry. For instance, the GUI may display microbiome fingerprint data that shows various characteristics of the microbiome fingerprint, dietary fingerprint data that shows various characteristics of the dietary fingerprint, microbiome ancestry data that shows various characteristics of the microbiome ancestry, recommendation data that identifies one or more recommendations relating to changing the microbiome of the user, and the like. In some configurations, the user may utilize an application 130 on the computing device 102 to interact with the nutritional environment. In some configurations, the application 130 may include functionality relating to processing at least a portion of the data 108.

As an example, the microbiome service 120 may provide recommendations generated by the nutritional service 132 to increase the diversity of foods eaten as there is no one good food for a microbiome. The recommendations may include to different gut-healthy foods, eat fermented foods, minimize highly processed foods (things like emulsifiers and artificial sweeteners may affect the microbiome). The microbiome service may base the recommendations on data obtained from the user, and other users.

The microbiome service 120 may also track the state of the microbiome of the user over time. For example, the microbiome service may provide data related to different microbiome analysis. In this way, the user may see how changes made by the user (e.g., eating different foods, changing exercise patterns, . . . ) have affected the microbiome.

FIG. 2 is a block diagram depicting an illustrative operating environment 200 in which a data ingestion service 110 receives and processes data associated with data associated with at home tests and sample collections. As illustrated in FIG. 2, the operating environment 200 includes the data ingestion service 110 that may be utilized in ingesting data utilized by the microbiome service 120.

In some configurations, the data manager 112 is configured to receive data such as, health data 202 that can include, but is not limited to microbiome data 206A, triglycerides data 206B, glucose data 206C, blood data 206D, wearable data 206E, questionnaire data 206F, psychological data (e.g., hunger, sleep quality, mood, . . . ) 206G, objective health data (e.g., height, weight, medical history, . . . ) 206H, nutritional data 140B, and other data 140C.

According to some examples, the microbiome data 206A includes data about the gut microbiome of an individual. The gut microbiome can host a large number of microbial species (e.g., >1000) that together have millions of genes. Microbial species include bacteria, fungi, parasites, viruses, and archaea. Imbalance of the normal gut microbiome has been linked with gastrointestinal conditions such as inflammatory bowel disease (IBD) and irritable bowel syndrome (IBS), and wider systemic manifestations of disease such as obesity and type 2 diabetes. The microbes of the gut undertake a variety of metabolic functions and are able to produce a variety of vitamins, synthesize essential and nonessential amino acids, and provide other functions. Amongst other functions, the microbiome of an individual provides biochemical pathways for the metabolism of non-digestible carbohydrates; some oligosaccharides that escape digestion; unabsorbed sugars and alcohols from the diet; and host-derived mucins.

The triglycerides data 206B may include data about triglycerides for an individual. In some examples, the triglycerides data 206B can be determined from an At Home Blood Test which in some cases is a finger prick on to a dried blood spot card. The glucose data 206C includes data about blood glucose. The glucose data 206C may be determined from various testing mechanisms, including at home measurements, such as a continuous glucose meter.

The blood data 206D may include blood tests relating to a variety of different biomarkers. As discussed above, at least some blood tests can be performed at home. In some configurations, the blood data 206D is associated with measuring blood sugar, insulin, c-peptides, triglycerides, IL-6 inflammation, ketone bodies, nutrient levels, allergy sensitivities, iron levels, blood count levels, HbA1c, and the like.

The wearable data 206E can include any data received from a computing device associated with an individual. For instance, an individual may wear an electronic data collection device 103, such as an activity-monitoring device, that monitors motion, heart rate, determines how much an individual has slept, the number of calories burned, activities performed, blood pressure, body temperature, and the like. The individual may also wear a continuous glucose meter that monitors blood glucose levels.

The questionnaire data 206F can include data received from one or more questionnaires, and/or surveys received from one or more individuals. The psychological data 206G, that may be subjectively obtained, may include data received from the individual and/or a computing device that generates data or input based on a subjective determination (e.g., the individual states that they are still hungry after a meal, or a device estimates sleep quality based on the movement of the user at night perhaps combined with heart rate data). The objective health data 206H includes data that can be objectively measured, such as but not limited to height, weight, medical history, and the like.

The nutritional data 140B can include data about food, which is referred to herein as “food data”. For example, the nutritional data can include nutritional information about different food(s) such as their macronutrients and micronutrients or the bioavailability of its nutrients under different conditions (raw vs cooked, or whole vs ground up). In some examples, the nutritional data 140C can include data about a particular food. For instance, before an individual consumes a particular meal, information about that food can be determined. As briefly discussed, the user might scan a barcode on the food item(s) being consumed and/or take one or more pictures of the food to determine the food, as well as the amount of food, being consumed.

The nutritional data can include food data that identifies foods consumed, a quantity of the foods consumed, food nutrition (e.g., obtained from a nutritional database), food state (e.g., cooked, reheated, frozen, etc.), food timing data (e.g., what time was the food consumed, how long did it take to consume, . . . ), and the like. The food state can be relevant for foods such as carbohydrates (e.g., pasta, bread, potatoes or rice), since carbohydrates may be altered by processes such as starch retrogradation. The food state can also be relevant for quantity estimation of the foods, since foods can change weight dramatically during cooking. In some instances, the user may also take a picture before and/or after consuming a meal to determine what food was consumed as well as how much of the food was consumed. The picture can also provide an indication as to the food state.

The other data 142B can include other data associated with the individual. For example, the other data 142B can include data that can be received directly from a computer application that logs information for an individual (e.g., food eaten, sleep, . . . ) and/or from the user via a user interface.

In some examples, different computing devices 102 associated with different users provide application data 204 to the data manager 112 for ingestion by the data ingestion service 110. As illustrated, computing device 102A provides app data 204A to the data manager 112, computing device 104B provides app data 204B to the data manager 112, and computing device 104N provides app data 204N to the data manager 112. There may be any number of computing devices utilized.

As discussed briefly above, the data manager 112 receives data from different data sources, processes the data when needed (e.g., cleans up the data for storage in a uniform manner), and stores the data within one or more data stores, such as the data store 140.

The data manager 112 can be configured to perform processing on the data before storing the data in the data store 140. For example, the data manager 112 may receive data for ketone bodies and then use that data to generate ketone body ratios. Similarly, the data manager 112 may process food eaten and generate meal calories, number of carbohydrates, fat to carbohydrate rations, how much fiber consumed during a time period, and the like. The data stored in the data store 140, or some other location, can be utilized by the microbiome service 120 to determine an accuracy of at home measurements of nutritional responses performed by users. The data outputted by the microbiome service 120 to the nutritional service may therefore contain different values than are stored in the data store 140, for example if a food quantity is adjusted.

FIGS. 3, 4, 5, 6, and 7 are flow diagrams showing processes 300, 400, 500, 600, and 700, respectively that illustrate aspects of generating microbiome fingerprints, dietary fingerprints, and microbiome ancestry data in accordance with examples described herein. It should be appreciated that at least some of the logical operations described herein with respect to FIGS. 3, 4, 5, 6, and 7, and the other FIGS., may be implemented (1) as a sequence of computer implemented acts or program modules running on a computing system and/or (2) as interconnected machine logic circuits or circuit modules within the computing system.

The implementation of the various components described herein is a matter of choice dependent on the performance and other requirements of the computing system. Accordingly, the logical operations described herein are referred to variously as operations, structural devices, acts, or modules. These operations, structural devices, acts, and modules may be implemented in software, in firmware, in special purpose digital logic and any combination thereof. It should also be appreciated that more or fewer operations may be performed than shown in the FIGS. and described herein. These operations may also be performed in parallel, or in a different order than those described herein.

FIG. 3 is a flow diagram showing a process 300 illustrating aspects of a mechanism disclosed herein for obtaining and utilizing microbiome data for a user to generate microbiome fingerprints, dietary fingerprints, and microbiome ancestry for users.

The process 300 may begin at 302, where microbiome sample/data is obtained from a user. As discussed above, a user may provide one or more microbiome samples that may be obtained at home or in a clinical setting. For example, the user may provide a sample or samples of their stool for microbiome analysis as part of the at home biological collection, and/or the sample(s) may be collected in a lab, or other clinical setting. In some configurations, the user may also provide other data that may be utilized when processing the sample. For instance, the user may provide timing data indicating when the sample was taken, conditions under which the sample was obtained, and/or other health data.

At 304, the microbiome data is processed. As discussed above, microbiome service 120 may generate DNA data from the sample. In some examples, the DNA is extracted from the cells of the microbiome sample and purified. Different techniques that are commercially available can be utilized for DNA extraction from the microbiome sample. Generally, the use of different extraction techniques may result in different biases that may affect an accurate microbial representation.

At 306, the microbial composition of the microbiome sample may be identified. According to some configurations, the microbiome service 120, or some other device or component, identifies the microbial composition of the microbiome (e.g., the species, genes, taxa, and the like). The microbiome service 120 may also generate reconstructed microbial genomes, determine a diversity of the microbiome, identify functions of the microbiome, identify a uniqueness of the microbiome, identify interesting species, and the like.

At 308, the diversity of the microbiome may be determined. As discussed above, the microbiome service 120 may determine the diversity of the microbiome associated with a user. In some examples, the diversity determined by the microbiome service 120 is the amount of individual bacteria from each of the bacterial species present in the microbiome. Having a more diverse microbiome may have health benefits. According to some configurations, the microbiome service 120 may provide this data, possibly along with recommendations, to the user via a UI, or some other interface.

At 310, reconstructed microbial genomes are generated. The microbiome service 120, or some other component or device may generate the reconstructed microbial genomes. Reconstruction of DNA fragments into genomes may utilize different techniques and methods and generally incorporates sequence assembly and sorting/clustering of assembled sequences into different bins associated with characteristic of a genome.

At 312, the functions of a microbiome may be determined. As discussed above, the microbiome service 120, or some other device or component, may determine the functions of a microbiome. Different techniques and methods may be utilized to determine the functions. Generally, the microbiome service 120 may map the sequencing reads against sequences of DNA (or amino acids) representing known genes (or proteins) and gene families (or protein families) to determine the functional potential of the microbiome.

At 314, other data associated with the microbiome of the user may be determined. As discussed above, the microbiome service 120, or some other device or component, may determine data such as the uniqueness of the microbiome (e.g., compared to the microbiome of other users), species identified as interesting, and the like.

At 316, the microbiome data associated with the user is stored. As discussed above, the microbiome service 120, or some other device or component, may store the microbiome data in a data store, such as user microbiome data 140A within data store 140.

At 318, the microbiome data associated with the user is utilized to generate microbiome fingerprints, dietary fingerprints, and microbiome ancestry for the user. As discussed above, the microbiome service 120, or some other device or component, may perform these tasks. See FIGS. 4-6 and related discussion for more details.

FIG. 4 is a flow diagram showing a process 400 illustrating aspects of a mechanism disclosed herein for generating a microbiome fingerprint for a user. As discussed above, the microbiome fingerprint may be generated using various techniques and methods. The following process is an example of generating a microbiome fingerprint.

At 402, microbiome data for a particular user is accessed. As discussed above, the microbiome service 120, or some other device or component, may access the microbiome data 140A within data store 140 to obtain the microbiome data for a user. In other examples, the microbiome data may be obtained/accessed using some other technique (e.g., accessing a different memory, receiving the data from some other source, such as data source(s) 150, and the like).

At 404, the microbiome data may be preprocessed to generate screened microbiome data. As discussed above, the microbiome service 120, or some other device or component, may process the sequencing data to generate screened sequencing data. The screened sequence data may make the generation of the different profiles described below be more accurate.

At 406, the quantitative taxonomic profiles are generated. As discussed above, the microbiome service 120, or some other device or component, may generate the quantitative taxonomic profiles. The quantitative taxonomic profiles can be obtained by mapping (i.e. matching the sequences) the sequencing reads against sequences representing the known microbial organisms. The mapping is then processed to produce relative abundances of the reference microbes. Many open source algorithms and corresponding implementations are available for this step, including for example, the techniques as described by Truong, Duy Tin, Eric A. Franzosa, Timothy L. Tickle, Matthias Scholz, George Weingart, Edoardo Pasolli, Adrian Tett, Curtis Huttenhower, and Nicola Segata. 2015. “MetaPhlAn2 for Enhanced Metagenomic Taxonomic Profiling.” Nature Methods 12 (10): 902-3 and the newer versions of the associated software, which are incorporated by reference herein in their entirety.

At 408, the quantitative functional potential profiles are generated. As discussed above, the microbiome service 120, or some other device or component, may generate the quantitative functional potential profiles. The quantitative functional potential profiles can be obtained by mapping the sequencing reads against sequences of DNA (or amino acids) representing known genes (or proteins) and gene families (or protein families). Based on the number of reads matching each gene or gene family the presence and abundance of the gene families and pathways are inferred. Several open source algorithms and corresponding implementations are available for this step, including for example the technique HUMAnN2 as described by Abubucker, Sahar, Nicola Segata, Johannes Goll, Alyxandria M. Schubert, Jacques Izard, Brandi L. Cantarel, Beltran Rodriguez-Mueller, et al. 2012. “Metabolic Reconstruction for Metagenomic Data and Its Application to the Human Microbiome.” PLoS Computational Biology 8 (6), Franzosa, E. A., McIver, L. J., Rahnavard, G., Thompson, L. R., Schirmer, M., Weingart, G., . . . Segata, N & Huttenhower, C. (2018). Species-level functional profiling of metagenomes and metatranscriptomes. Nature methods, 15(11), 962, and any newer versions of the associated software, which are incorporated by reference herein in their entirety.

At 410, the strain-level genomic profiles are generated. As discussed above, the microbiome service 120, or some other device or component, may generate the strain-level genomic profiles. The strain-level genomic profiles, or the third descriptor, can be obtained with reference-based and assembly-based approaches. For reference-based approaches the methods use specific genetic markers against which the reads are mapped, and single-nucleotide polymorphisms are inferred. The combinations of single-nucleotide polymorphisms provide strain-specific profiles. Some open source algorithms and implementations for this step are available, including for example the techniques described by Truong, Duy Tin, Adrian Tett, Edoardo Pasolli, Curtis Huttenhower, and Nicola Segata. 2017. “Microbial Strain-Level Population Structure and Genetic Diversity from Metagenomes.” Genome Research 27 (4): 626-38, which is incorporated by reference herein in its entirety. In assembly-based approaches, reads may be first concatenated to form longer contiguous sequences such as described by Li, Dinghua, Chi-Man Liu, Ruibang Luo, Kunihiko Sadakane, and Tak-Wah Lam. 2015. “MEGAHIT: An Ultra-Fast Single-Node Solution for Large and Complex Metagenomics Assembly via Succinct de Bruijn Graph.” Bioinformatics 31 (10): 1674-76, which is incorporated by reference herein in its entirety.

Contigs may then be clustered in bins representing the sequences of whole genomes, such as described by Kang, Dongwan D., Feng Li, Edward Kirton, Ashleigh Thomas, Rob Egan, Hong An, and Zhong Wang. 2019. “MetaBAT 2: An Adaptive Binning Algorithm for Robust and Efficient Genome Reconstruction from Metagenome Assemblies.” PeerJ 7 (July): e7359, which is incorporated by reference herein in its entirety. The resulting draft genomes may be quality controlled using for example the techniques described by Parks, Donovan H., Michael Imelfort, Connor T. Skennerton, Philip Hugenholtz, and Gene W. Tyson. 2015. “CheckM: Assessing the Quality of Microbial Genomes Recovered from Isolates, Single Cells, and Metagenomes” Genome Research 25 (7): 1043-55, which is incorporated by reference herein in its entirety. The quality-controlled genomes represent single strains in the microbiome.

At 412, the microbiome fingerprint for the user is generated. As discussed above, the microbiome service 120, or some other device or component, may combine the data associated with the different indexes generated at 406, 408, and 410 to generate the microbiome fingerprint for the user.

At 414, the microbiome fingerprint may be utilized. As discussed above, the microbiome service 120, or some other device or component, may utilize the microbiome fingerprint when generating the dietary fingerprint, the microbiome ancestry, generating recommendations for the user (e.g., nutritional), and/or some other task.

FIG. 5 is a flow diagram showing a process 500 illustrating aspects of a mechanism disclosed herein for generating a dietary fingerprint for a user.

The process 500 may begin at 502, where microbiome data for a particular user are accessed. As discussed above, the microbiome service 120, or some other device or component, may access the microbiome data 140A within data store 140 to obtain the microbiome data for a user. In other examples, the microbiome data may be obtained/accessed using some other technique (e.g., accessing a different memory, receiving the data from some other source, such as data source(s) 150, and the like).

At 504, dietary fingerprint data is generated. As discussed above, the microbiome service 120, or some other device or component, may generate dietary fingerprint data that identifies a similarity between the microbiome of a particular user and a “dietary fingerprint” is data that identifies how the microbiome of a user is associated with one or more different indexes. The indexes may include, but are not limited to a Mediterranean diet index, a vegetarian diet index, a fast food index, an internal fat index, a fat-digesting index, a carbohydrate-digesting index, a health index, a fasting index, a ketogenic index, and the like. According to some configurations, one or more computers of a microbiome service generate a score, such as from 0-100, (or some other indicator) that indicates how closely the microbiome of the user is associated with a particular index.

As an example, the Mediterranean diet index score for a user indicates how closely the microbiome of the user resembles the typical microbiome of someone on a Mediterranean diet. The vegetarian diet index score indicates how closely the microbiome of the user resembles someone on a vegetarian diet. The fast food index score indicates how closely the microbiome of the user resembles someone on a fast food diet. The internal fat index score indicates how closely the microbiome of the user resembles someone with high or low visceral fat. The fat-digesting index score indicates how closely the microbiome of the user resembles someone with low post-prandial triacylglycerol (TAG) rises. The carbohydrate-digesting index score indicates how closely the microbiome of the user resembles someone with low post-prandial glucose rises. The health index score indicates how closely the microbiome of the user resembles someone that is healthy. The fasting index score indicates how closely the microbiome of the user resembles someone that fasts regularly. The ketogenic index score indicates how closely the microbiome of the user resembles someone who is ketogenic.

At 506, a determination is made as to whether another dietary index is to be compared. As discussed above, there may be a variety of dietary indexes, including but not limited to a Mediterranean diet index, a vegetarian diet index, a fast food index, an internal fat index, a fat-digesting index, a carbohydrate-digesting index, a health index, a fasting index, a ketogenic index, and the like. When there is another index, the process 500 returns to 504. When there is not another index, the process 500 moves to 508.

At 508, the dietary index(es) associated with the user are identified. As discussed above, the microbiome service 120, or some other device or component, may identify one or more diets that resemble the microbiome of the user. In some examples, the microbiome service 120 identifies the closest dietary index (e.g., based on a score). In other examples, the microbiome service 120 may rank the dietary index.

At 510, the dietary fingerprint data may be utilized. As discussed above, the microbiome service 120, or some other device or component, may utilize the dietary fingerprint data when providing data to the user, when generating the microbiome ancestry data, generating recommendations for the user (e.g., nutritional), and/or performing some other task.

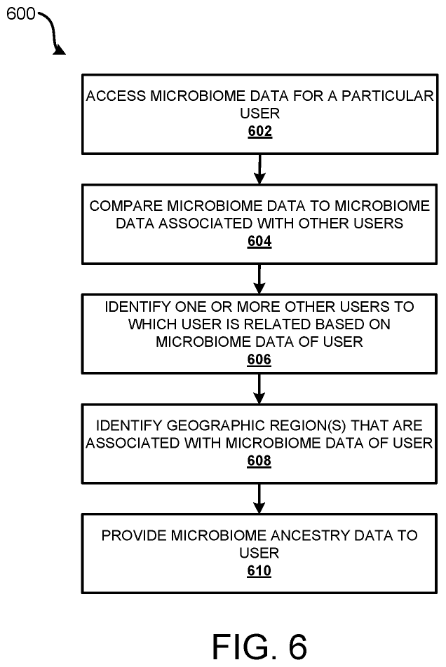

FIG. 6 is a flow diagram showing a process 600 illustrating aspects of a mechanism disclosed herein for generating a microbiome ancestry for a user.

The process 600 may begin at 602, where microbiome data for a particular user is accessed. As discussed above, the microbiome service 120, or some other device or component, may access the microbiome data 140A within data store 140 to obtain the microbiome data for a user. In other examples, the microbiome data may be obtained/accessed using some other technique (e.g., accessing a different memory, receiving the data from some other source, such as data source(s) 150, and the like).

At 604, the microbiome data is compared to microbiome data from other users. As discussed above, the microbiome service 120, or some other device or component, may utilize the microbiome data, such as the microbiome fingerprint data of a particular user, and compare microbiome fingerprint data of other users. According to some configurations, the microbiome service 120 may generate one or more indicators that identify how close another user is to the user based on a similarity of the microbiome data.

At 606, one or more other users are identified based on a similarity of the microbiome data between the users. As discussed above, the microbiome service 120, or some other device or component, may identify the related users based on a score generate the microbiome service 120, or some other indicators.

At 608, the geographic region(s) that are commonly associated with the microbiome data of a user are identified. As discussed above, the microbiome service 120, or some other device or component, may identify that different geographic regions are more closely linked to certain microbiomes.

At 610, the microbiome ancestry data may be utilized. As discussed above, the microbiome service 120, or some other device or component, may utilize the microbiome ancestry data when providing data to the user, when generating the microbiome ancestry data, generating recommendations for the user (e.g., nutritional), and/or performing some other task.

FIG. 7 is a flow diagram showing a process 700 illustrating aspects of a mechanism disclosed herein for obtaining test data, including microbiome data, to be utilized for generating microbiome fingerprints, dietary fingerprints, and microbiome ancestry for users.

At 702, food(s) for at home measurements of nutritional responses may be selected. As briefly discussed above, different foods may be selected for a user to eat before a test is performed in order to evoke a desired response. The foods can include foods for a series of standardized meals, a single food, or some other combination of foods.

At 704, food data is received. As discussed above, the food data is associated with foods that are utilized to evoke a nutritional response. The food data can include foods for a series of standardized meals, a single food, or some other combination of foods. The food data can include data such as foods consumed, a quantity of the foods consumed, food nutrition (e.g., obtained from a nutritional database), food state (e.g., cooked, reheated, frozen, etc.), food timing data (e.g., what time was the food consumed, how long did it take to consume, . . . ), and the like. The food state can be relevant for foods such as carbohydrates (e.g., pasta, bread, potatoes or rice), since carbohydrates may be altered by processes such as starch retrogradation. The food state can also be relevant for quantity estimation of the foods, since foods can change weight dramatically during cooking.

At 706, at home test(s) are performed. The tests may include at home tests as described above and/or the collection of one or more samples (e.g., stool for microbiome analysis).

At 708, test data associated with the at home tests including microbiome data is received. As discussed above, microbiome data may be associated with one or more tests. In some configurations, the microbiome data includes a stool sample, timing data for the sample (e.g., when collected, how long stored before providing to a lab), data associated with collection of the sample (e.g., how was sample stored, was the sample contaminated), as well as other data. For example, a user may be instructed to take a picture of the sample and provide the image to the service.

At 710, the test data is utilized to generate microbiome fingerprints, dietary fingerprints, and microbiome ancestry. In some examples, the test data is used by the microbiome service 120 to generate the microbiome fingerprints, dietary fingerprints, and microbiome ancestry. The nutritional service 132 may also use the test data to generate nutritional recommendations that are personalized for a particular user.

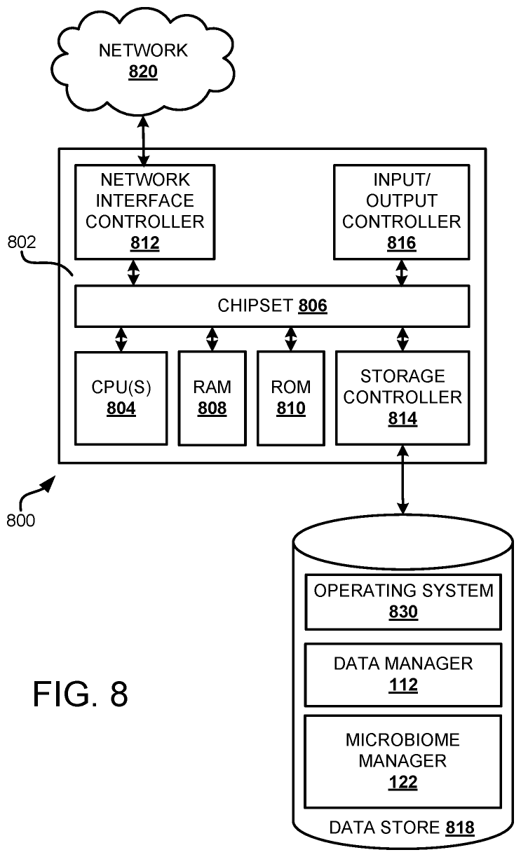

FIG. 8 shows an example computer architecture for a computer 800 capable of executing program components for generating microbiome fingerprints, dietary fingerprints, and microbiome ancestry for users in the manner described above. The computer architecture shown in FIG. 8 illustrates a conventional server computer, workstation, desktop computer, laptop, tablet, network appliance, digital cellular phone, smart watch, or other computing device, and may be utilized to execute any of the software components presented herein. For example, the computer architecture shown in FIG. 8 may be utilized to execute software components for performing operations as described above. The computer architecture shown in FIG. 8 might also be utilized to implement a computing device 102, or any other of the computing systems described herein.

The computer 800 includes a baseboard 802, or “motherboard,” which is a printed circuit board to which a multitude of components or devices may be connected by way of a system bus or other electrical communication paths. In one illustrative example, one or more central processing units (“CPUs”) 804 operate in conjunction with a chipset 806. The CPUs 804 may be standard programmable processors that perform arithmetic and logical operations necessary for the operation of the computer 800.

The CPUs 804 perform operations by transitioning from one discrete, physical state to the next through the manipulation of switching elements that differentiate between and change these states. Switching elements may generally include electronic circuits that maintain one of two binary states, such as flip-flops and electronic circuits that provide an output state based on the logical combination of the states of one or more other switching elements, such as logic gates. These basic switching elements may be combined to create more complex logic circuits, including registers, adders-subtractors, arithmetic logic units, floating-point units and the like.

The chipset 806 provides an interface between the CPUs 804 and the remainder of the components and devices on the baseboard 802. The chipset 806 may provide an interface to a RAM 808, used as the main memory in the computer 800. The chipset 806 may further provide an interface to a computer-readable storage medium such as a read-only memory (“ROM”) 810 or non-volatile RAM (“NVRAM”) for storing basic routines that help to startup the computer 800 and to transfer information between the various components and devices. The ROM 810 or NVRAM may also store other software components necessary for the operation of the computer 800 in accordance with the examples described herein.

The computer 800 may operate in a networked environment using logical connections to remote computing devices and computer systems through a network, such as the network 820. The chipset 806 may include functionality for providing network connectivity through a network interface controller (“NIC”) 812, such as a mobile cellular network adapter, WiFi network adapter or gigabit Ethernet adapter. The NIC 812 is capable of connecting the computer 800 to other computing devices over the network 820. It should be appreciated that multiple NICs 812 may be present in the computer 800, connecting the computer to other types of networks and remote computer systems.

The computer 800 may be connected to a mass storage device 818 that provides non-volatile storage for the computer. The mass storage device 818 may store system programs, application programs, other program modules and data, which have been described in greater detail herein. The mass storage device 818 may be connected to the computer 800 through a storage controller 814 connected to the chipset 806. The mass storage device 818 may consist of one or more physical storage units. The storage controller 814 may interface with the physical storage units through a serial attached SCSI (“SAS”) interface, a serial advanced technology attachment (“SATA”) interface, a fiber channel (“FC”) interface, or other type of interface for physically connecting and transferring data between computers and physical storage units.

The computer 800 may store data on the mass storage device 818 by transforming the physical state of the physical storage units to reflect the information being stored. The specific transformation of physical state may depend on various factors, in different implementations of this description. Examples of such factors may include, but are not limited to, the technology used to implement the physical storage units, whether the mass storage device 818 is characterized as primary or secondary storage and the like.

For example, the computer 800 may store information to the mass storage device 818 by issuing instructions through the storage controller 814 to alter the magnetic characteristics of a particular location within a magnetic disk drive unit, the reflective or refractive characteristics of a particular location in an optical storage unit, or the electrical characteristics of a particular capacitor, transistor, or other discrete component in a solid-state storage unit. Other transformations of physical media are possible without departing from the scope and spirit of the present description, with the foregoing examples provided only to facilitate this description. The computer 800 may further read information from the mass storage device 818 by detecting the physical states or characteristics of one or more particular locations within the physical storage units.

In addition to the mass storage device 818 described above, the computer 800 may have access to other computer-readable storage media to store and retrieve information, such as program modules, data structures, or other data. It should be appreciated by those skilled in the art that computer-readable storage media is any available media that provides for the non-transitory storage of data and that may be accessed by the computer 800.

By way of example, and not limitation, computer-readable storage media may include volatile and non-volatile, removable and non-removable media implemented in any method or technology. Computer-readable storage media includes, but is not limited to, RAM, ROM, erasable programmable ROM (“EPROM”), electrically-erasable programmable ROM (“EEPROM”), flash memory or other solid-state memory technology, compact disc ROM (“CD-ROM”), digital versatile disk (“DVD”), high definition DVD (“HD-DVD”), BLU-RAY, or other optical storage, magnetic cassettes, magnetic tape, magnetic disk storage or other magnetic storage devices, or any other medium that can be used to store the desired information in a non-transitory fashion.

The mass storage device 818 may store an operating system 830 utilized to control the operation of the computer 800. According to one example, the operating system comprises the LINUX operating system. According to another example, the operating system comprises the WINDOWS® SERVER operating system from MICROSOFT Corporation. According to another example, the operating system comprises the iOS operating system from Apple. According to another example, the operating system comprises the Android operating system from Google or its ecosystem partners. According to further examples, the operating system may comprise the UNIX operating system. It should be appreciated that other operating systems may also be utilized. The mass storage device 818 may store other system or application programs and data utilized by the computer 800, such as components that include the data manager 122, the microbiome manager 122 and/or any of the other software components and data described above. The mass storage device 818 might also store other programs and data not specifically identified herein.

In one example, the mass storage device 818 or other computer-readable storage media is encoded with computer-executable instructions that, when loaded into the computer 800, create a special-purpose computer capable of implementing the examples described herein. These computer-executable instructions transform the computer 800 by specifying how the CPUs 804 transition between states, as described above. According to one example, the computer 800 has access to computer-readable storage media storing computer-executable instructions which, when executed by the computer 800, perform the various processes described above with regard to FIGS. 4-8. The computer 800 might also include computer-readable storage media for performing any of the other computer-implemented operations described herein.

The computer 800 may also include one or more input/output controllers 816 for receiving and processing input from a number of input devices, such as a keyboard, a mouse, a touchpad, a touch screen, an electronic stylus, or other type of input device. Similarly, the input/output controller 816 may provide output to a display, such as a computer monitor, a flat-panel display, a digital projector, a printer, a plotter, or other type of output device. It will be appreciated that the computer 800 may not include all of the components shown in FIG. 8, may include other components that are not explicitly shown in FIG. 8, or may utilize an architecture completely different than that shown in FIG. 8.

Based on the foregoing, it should be appreciated that technologies for generating microbiome fingerprints, dietary fingerprints, and microbiome ancestry have been presented herein. Moreover, although some of the subject matter presented herein has been described in language specific to computer structural features, methodological acts and computer readable media, it is to be understood that the invention defined in the appended claims is not necessarily limited to the specific features, acts, or media described herein. Rather, the specific features, acts and media are disclosed as example forms of implementing at least some of the claims.

The subject matter described above is provided by way of illustration only and should not be construed as limiting. Furthermore, the claimed subject matter is not limited to implementations that solve any or all disadvantages noted in any part of this disclosure. Various modifications and changes may be made to the subject matter described herein without following the examples and applications illustrated and described, and without departing from the true spirit and scope of the present invention, which is set forth in the following claims.

What is claimed is:

1. A method, comprising:
accessing microbiome data associated with a user;
generating a quantitative taxonomic profile from the microbiome data;
generating a quantitative functional potential profile from the microbiome data;
generating a strain-level genomic profile from the microbiome data;
generating, based on the quantitative taxonomic profile, the quantitative functional potential profile, and the strain-level genomic profile, a microbiome fingerprint that identifies a microbiome of the user at a particular point in time; and
perform one or more actions utilizing the microbiome fingerprint, wherein the one or more actions include one or more of providing data associated with the microbiome fingerprint to a computing device associated with the user, generating a dietary fingerprint, and generating microbiome ancestry data.

2. The method of claim 1, wherein generating the dietary fingerprint includes:
performing a first comparison that compares the microbiome fingerprint with one or more second microbiome fingerprints that are representative of at least one other user that is indicated to follow a first diet;
performing a second comparison that compares the microbiome fingerprint with one or more third microbiome fingerprints that are representative of at least one other user that is indicated to follow a second diet; and
determining that the microbiome fingerprint of the user is similar to the first diet or the second diet based on the first comparison and the second comparison.

3. The method of claim 2, wherein the first diet is selected from one or more of a Mediterranean diet, a vegetarian diet, a fast food diet, and a ketogenic diet, and wherein the second diet is different from the first diet.

4. The method of claim 2, further comprising performing a third comparison that compares the microbiome fingerprint with a fourth microbiome fingerprint that is representative of at least one other user that is indicated to exhibit a characteristic, wherein the characteristic is selected from at least one of an internal fat characteristic, a fat-digesting characteristic, a carbohydrate-digesting characteristic, a gut transit time index, or a health characteristic.

5. The method of claim 1, wherein the one or more actions includes generating a microbiome ancestry data for the user, wherein generating the dietary fingerprint includes performing a comparison that compares the microbiome fingerprint with other fingerprints that are associated with different users; and determining that one or more of the different users are similar to the user based on the comparison.

6. The method of claim 1, wherein the one or more actions includes identifying that the microbiome fingerprint is associated with a geographic region, wherein the identifying includes comparing the microbiome fingerprint with other microbiome fingerprints associated with different users in different geographic regions.

7. The method of claim 1, further comprising generating a graphical user interface that includes user interface elements, causing the graphical user interface to be presented on a display associated with the user, and causing data associated with the microbiome fingerprint to be presenting on the display.

8. The method of claim 1, further comprising generating a nutritional recommendation based on the microbiome fingerprint and causing the nutritional recommendation to be provided to a computing device associated with the user.

9. A system, comprising:
a data ingestion service, including one or more processors, configured to
access data associated with a microbiome of a user; and
a microbiome service, including one or more processors, configured to
generate, based at least in part on the data, microbiome data that includes a microbiome fingerprint associated with the user, wherein the microbiome fingerprint identifies the microbiome of the user; and
cause one or more actions to be performed, wherein the one or actions include one or more of generating a dietary fingerprint using at least a portion of the microbiome data and generating microbiome ancestry data using at least a second portion of the microbiome data.

10. The system of claim 9, wherein generating the microbiome data includes:
generating a quantitative taxonomic profile from the data;
generating a quantitative functional potential profile from the data; and
generating a strain-level genomic profile from the data.

11. The system of claim 9, wherein generating the dietary fingerprint includes:
generating a first score that indicates a similarity between at least a portion of the microbiome data with second microbiome data that is representative of at least one other user that is indicated to one or more of follow a first diet and exhibit a first health characteristic;
generating a second score that indicates a similarity between at least a portion of the microbiome data with third microbiome data that is representative of at least one other user that is indicated to one or more of follow a second diet and exhibit a second health characteristic; and
associating the user with one or more of the first diet, the second diet, the first health characteristic, and the second health characteristic based, at least in part, on the first score and the second score.

12. The system of claim 11, wherein the first diet, the second diet, the first health characteristic, and the second health characteristic are selected from one or more of a Mediterranean diet, a vegetarian diet, a fast food diet, a ketogenic diet, an internal fat characteristic, a fat-digesting characteristic, a carbohydrate-digesting characteristic, and a health characteristic.

13. The system of claim 9, wherein generating the microbiome ancestry data includes identifying that the microbiome fingerprint is similar to at least one second microbiome fingerprint that is determined from one or more different users.

14. The system of claim 9, wherein generating the microbiome ancestry data includes identifying that the microbiome fingerprint is associated with a geographic region, wherein the identifying includes comparing the microbiome fingerprint with other microbiome fingerprints associated with different users in different geographic regions.

15. The system of claim 9, further comprising generating a nutritional recommendation based, at least in part, on the microbiome fingerprint and causing the nutritional recommendation to be provided to a computing device associated with the user.

16. The system of claim 9, further comprising a nutritional service configured to generate a national recommendation based, at least in part, on the microbiome data.

17. A non-transitory computer-readable storage medium having computer-executable instructions stored thereupon which, when executed by a computer, cause the computer to:
access data associated with a microbiome of a user;
generate, based at least in part on the data, a microbiome fingerprint that identifies the microbiome of the user; and
cause one or more actions to be performed, wherein the one or actions include one or more of generating a dietary fingerprint using the microbiome fingerprint and generating microbiome ancestry data using the microbiome fingerprint.

18. The non-transitory computer-readable storage medium of claim 17, wherein generating the microbiome fingerprint includes:
generating a quantitative taxonomic profile associated with a microbiome sample of the user;
generating a quantitative functional potential profile associated with a microbiome sample of the user; and
generating a strain-level genomic profile associated with a microbiome sample of the user.

19. The non-transitory computer-readable storage medium of claim 17, wherein generating the dietary fingerprint includes:
generating a first indication of a similarity between the microbiome fingerprint and microbiome data associated with one or more second microbiome fingerprints, wherein the microbiome data is representative of at least one other user that is indicated to one or more of follow a first diet and exhibit a first health characteristic;
generating a second indication of a similarity between the microbiome fingerprint and second microbiome data associated with one or more third microbiome fingerprints, wherein the second microbiome data is representative of at least one other user that is indicated to one or more of follow a second diet and exhibit a second health characteristic; and
associating the user with one or more of the first diet, the second diet, the first health characteristic, and the second health characteristic based, at least in part, on the first score and the second score,
wherein the first diet, the second diet, the first health characteristic, and the second health characteristic are selected from one or more of a Mediterranean diet, a vegetarian diet, a fast food diet, a ketogenic diet, an internal fat characteristic, a fat-digesting characteristic, a carbohydrate-digesting characteristic, and a health characteristic.

20. The non-transitory computer-readable storage medium of claim 17, wherein generating the microbiome ancestry data includes:
identifying that the microbiome fingerprint is similar to at least one second microbiome fingerprint that is determined from one or more different users; and
identifying that the microbiome fingerprint is associated with a geographic region, wherein the identifying includes comparing the microbiome fingerprint with other microbiome fingerprints associated with different users in different geographic regions.
